(19) United States
(12) Patent Application Publication
(10) Pub. No.: US 2026/0083695 A1
(43) Pub. Date: Mar. 26, 2026

(54) MITIGATION OF VEISALGIA AND NAUSEA AND METHODS THEREOF

(71) Applicant: April Hang, Duluth, GA (US)

(72) Inventor: April Hang, Duluth, GA (US)

(21) Appl. No.: 19/324,515

(22) Filed: Sep. 10, 2025

Related U.S. Application Data
(60) Provisional application No. 63/698,393, filed on Sep. 24, 2024.

(51) Int. Cl. A61K 31/198; A61K 31/164; A61K 31/185; A61K 31/191; A61K 31/194; A61K 31/353; A61K 31/375; A61K 31/455; A61K 31/525; A61K 31/60; A61K 31/675; A61K 31/714; A61K 33/08; A61K 33/42; A61K 36/28; A61K 36/534; A61K 36/752; A61K 36/9068

(52) U.S. Cl. CPC A61K 31/198; A61K 31/164; A61K 31/185; A61K 31/191; A61K 31/194; A61K 31/353; A61K 31/375; A61K 31/455; A61K 31/525; A61K 31/60; A61K 31/675; A61K 31/714; A61K 33/08; A61K 33/42; A61K 36/28; A61K 36/534; A61K 36/752; A61K 36/9068

(57) ABSTRACT

An orally deliverable composition for reducing veisalgia symptoms and nausea in a subject includes dihydromyricetin, acetylcysteine, and an antiemetic that decreases smooth muscle contraction in the digestive tract. The antiemetic is selected from the group consisting of phosphoric acid, ginger, peppermint, lemon, bismuth subsalicylate, and combinations thereof in an amount sufficient to decrease smooth muscle contraction and inhibit nausea in the subject. The composition is packaged as a viscous liquid, capsule, tablet or chewable form for oral administration. The composition preferably contains no free form taurine amino acid to avoid taurine-induced nausea. The composition may contain neuroprotective forms of taurine such as magnesium acetyl taurate which is not known to cause nausea in subjects and which can pass the blood brain barrier to protect the brain from some effects of veisalgia.

### FIELD OF THE INVENTION

The present invention relates to methods and compositions for managing symptoms of veisalgia (commonly known as hangover). More particularly, the invention provides compositions that effectively manage veisalgia symptoms while specifically mitigating nausea in vivo.

### BACKGROUND OF THE INVENTION

The symptoms caused by the moderate or excessive intake of alcohol are unpleasant, particularly in the post-exhilaration or veisalgia stage (i.e., “hangover”). Such symptoms include, for example, substantial physical and mental fatigue, nausea, loss of appetite, tremors of the hand and limbs (the “shakes”), joint weakness/pain, dehydration, irritability, lack of coordination, concentration difficulties, sleeplessness, impaired memory and visual-spatial skills, headache, drowsiness, dry mouth, dizziness, gastro-intestinal complaints, sweating, and anxiety.

Hangover symptoms typically develop when BAC returns to zero. It is not known why these symptoms are present after alcohol and its metabolites are eliminated from the body. Excessive consumption of alcohol is defined as attaining blood alcohol content (BAC) of 0.055% and above. However, even moderate drinking (BAC below 0.055%) can also cause hangover symptoms in some people. There is a need for a more effective and accessible manner by which to manage the symptoms of veisalgia.

Common remedies include the amino acid taurine for improving hydration and stabilizing cell membranes. Taurine, in dosages of 1500 mg per day works well for improving hydration and helps with many ailments. Unfortunately, it can cause nausea when consumed orally in its isolated or free form.

The nausea often occurs while this amino acid passes through the digestive tract. Specifically, free form taurine supplementation is associated with gastrointestinal discomfort, including nausea, vomiting, stomach pain, and diarrhea, particularly when taken in higher doses or on an empty stomach. This side effect arises because taurine can irritate the digestive tract, potentially altering gut motility or stomach acid production, leading to these adverse reactions.

For individuals already experiencing nausea as a primary symptom of veisalgia, the inclusion of free taurine in remedies exacerbates the problem, counteracting the intended relief. Studies and reports on taurine supplementation indicate that while it is generally well-tolerated, digestive issues like nausea are among the most reported side effects, especially in supplemental forms found in energy drinks or isolated supplements. This underscores the need to avoid free form taurine in hangover remedies to prevent compounding nausea, while exploring alternative bound or encapsulated forms that deliver taurine's benefits without these drawbacks.

What is desired is a way of minimizing hangover symptoms, while also minimizing nausea caused by the veisalgia remedy.

### SUMMARY OF INVENTION

The invention includes compositions and methods for preventing, inhibiting, reducing, and/or shortening the duration of symptoms associated with the consumption of alcohol and/or hangover. In particular, the invention described herein relates to administering to a subject an effective amount of a composition described herein prior to, during, or soon after alcohol intake to prevent, inhibit, reduce, and/or shorten the duration of symptoms associated with consumption of alcohol and/or hangover.

In a preferred embodiment, the invention is an orally deliverable composition that reduces veisalgia symptoms and nausea in a subject. The composition includes dihydromyricetin, acetylcysteine, and an antiemetic that decreases smooth muscle contraction in the digestive tract.

The antiemetic is selected from the group consisting of phosphoric acid, ginger, peppermint, lemon, bismuth subsalicylate, and combinations thereof in an amount sufficient to decrease smooth muscle contraction and inhibit nausea in the subject. The composition is packaged as a viscous liquid, capsule, tablet or chewable form for oral administration.

The composition preferably contains no free form taurine amino acid to avoid taurine-induced nausea.

As an alternative to the use of magnesium glycinate as a primary source of magnesium, the magnesium can be selected from the group consisting of: magnesium oxide, magnesium citrate, magnesium glycinate, magnesium acetyl taurate, magnesium sulfate, magnesium L-Threonate; magnesium salts, and combinations thereof.

In a preferred embodiment, an amount of magnesium acetyl taurate, which is not liposomally encapsulated, is used to sooth cramped muscles, offer neuroprotection, and improve cellular hydration. Thus, the composition may include neuroprotective forms of taurine such as magnesium acetyl taurate which is not known to cause nausea in subjects and which can pass the blood brain barrier to protect the brain and neurons from some effects of veisalgia.

The magnesium, in another embodiment, includes magnesium acetyl taurate which is liposomally encapsulated to offer neuroprotection to the brain and to bypass the upper digestive tract.

The composition in another embodiment, includes magnesium glycinate as a primary magnesium source in an amount between 25 mg and 300 mg, combined with a secondary magnesium source which is magnesium acetyl taurate to increase brain tissue levels of magnesium and to provide neuroprotection to brain tissue without nausea, which can be induced by free form taurine supplementation.

The milk thistle is provided in an amount up to about 420 mg, and preferably within the range of 100 mg to 400 mg.

### DETAILED DESCRIPTION OF EMBODIMENTS OF THE INVENTION

The present invention is also directed to methods, and pharmaceutical compositions, to reduce and/or prevent the symptoms associated with moderate and excessive intake of alcohol. After intake of alcohol, many people develop symptoms referred to as “hangover” symptoms or veisalgia. These symptoms include, for example, substantial physical and mental fatigue, drowsiness, nausea, loss of appetite, tremors of the hand and limbs (the “shakes”), joint weakness/pain, dehydration, dry mouth (i.e., thirst), irritability, anxiety, lack of coordination, dizziness, difficulty in concentration, sleep disturbances (e.g., sleeplessness), impaired memory and visual-spatial skills, gastro-intestinal complaints, sweating and headache.

In some embodiments, the methods include the administration of the compositions disclosed herein prior to the onset of such symptoms. In some embodiments, the methods include the administration of the compositions disclosed herein while consuming alcohol. In some embodiments, the methods include the administration of the compositions disclosed herein prior to consuming alcohol.

In some embodiments, the compositions disclosed herein comprise acetylcysteine, ascorbic acid, caffeine, methylcobalamin, pyridoxal-5-phosphate, riboflavin, magnesium glycinate, calcium L-5-methyl-tetrahydrofolate, milk thistle, panthenol, taurine, DHM dihydromyricetin, and/or niacinamide. The ingredients disclosed herein can be supplied in combination as one-unit dose. It is surprising that the compositions disclosed herein prevent, inhibit, reduce, and/or shorten the duration of the symptoms of hangover. The components of the compositions disclosed herein when taken in combination, prior to, during, or shortly after alcohol consumption, have surprisingly been found to prevent, inhibit, reduce, and/or shorten the duration of symptoms of hangover.

The methods disclosed herein comprise the administration of the compositions disclosed herein to a human in an amount which is effective to inhibit the symptoms occurring after moderate or excessive alcohol intake.

In the present specification, the term “inhibit” includes “reduce” and/or “prevent” and/or “shorten duration.” That is, the method of the present invention is considered to be effective if it causes one or more of: a reduction/inhibition/prevention of any symptom associated with veisalgia and/or shortening of the duration of an episode of any such symptom.

Reduction of symptoms can be assessed by comparing the magnitude and/or duration of at least one hangover symptom in a subject at two different occasions, for example, in some embodiments, i) when administered a composition disclosed herein, after which the subject intakes alcohol; and ii) when not administered a composition disclosed herein, and the subject intakes substantially the same amount of alcohol. The method disclosed herein is considered to be effective if it reduces/inhibits/prevents/shortens the duration of headache and/or fatigue and/or thirst. In some embodiments, hangover symptoms are reduced by at least about 10%, at least about 20%, at least about 30%, at least about 40%, at least about 50%, at least about 60%, at least about 70%, at least about 80%, at least about 90%, or at least about 100%.

Throughout this specification, quantities are defined by ranges, and by lower and upper boundaries of ranges. Each lower boundary can be combined with each upper boundary to define a range. The lower and upper boundaries should each be taken as a separate element.

The actual preferred amounts of composition will vary according to the particular compositions formulated, the mode of application, the particular sites of application, and the subject being treated (e.g., age, gender, size, tolerance to drug, etc.). In some embodiments, the compositions are administered as a single dose. In some embodiments, a single dose is at least one unit (e.g., pill, capsule, tablet, chewing gum, transdermal patch, etc.). In some embodiments, a single dose is more than one unit, for example, more than one pill, capsule or tablet.

In a preferred embodiment, the invention is an orally deliverable composition that reduces veisalgia symptoms and nausea in a subject. The composition includes dihydromyricetin, acetylcysteine, and an antiemetic that decreases smooth muscle contraction in the digestive tract.

The antiemetic is selected from the group consisting of phosphoric acid, ginger, peppermint, lemon, bismuth subsalicylate, and combinations thereof in an amount sufficient to decrease smooth muscle contraction and inhibit nausea in the subject.

The composition is packaged as a viscous liquid, capsule, tablet, liquid gel capsule, or chewable form for oral administration.

The composition preferably contains no free form taurine amino acid to avoid taurine-induced nausea.

The composition may contain neuroprotective forms of taurine such as magnesium acetyl taurate which is not known to cause nausea in subjects, and which can pass the blood brain barrier to protect the brain from some effects of veisalgia.

Magnesium is a vital nutrient for regulating many body processes, including muscle and nerve function, blood sugar levels, and blood pressure.

The biological mechanism of action of magnesium glycinate primarily stems from magnesium's role as an essential cofactor in over 300 enzymatic reactions throughout the body. Magnesium facilitates energy production by participating in the synthesis and utilization of adenosine triphosphate (ATP), the primary energy currency of cells, through its involvement in glycolysis, the Krebs cycle, and oxidative phosphorylation. It also regulates ion channels, particularly calcium and potassium channels, which are critical for neuromuscular transmission, muscle contraction and relaxation, and maintaining cardiac rhythm.

Additionally, magnesium supports protein synthesis, DNA and RNA stability, and neurotransmitter release, contributing to nerve function and reducing excitotoxicity in the brain. In the context of veisalgia, alcohol consumption can deplete magnesium stores by increasing urinary excretion and impairing absorption, leading to symptoms such as muscle cramps, fatigue, headaches, and irritability; supplementing with magnesium glycinate helps replenish these levels, promoting cellular hydration, reducing inflammation, and supporting recovery without exacerbating nausea due to its gentle digestive profile. In some embodiments, the magnesium glycinate is provided in an amount up to 300 mg or from about 25 mg to about 125 mg.

In various embodiments, the magnesium can be selected from the group consisting of: magnesium oxide, magnesium citrate, magnesium glycinate, magnesium acetyl taurate, magnesium sulfate, magnesium L-Threonate; magnesium salts, and combinations thereof.

In one embodiment of the invention, a primary form of magnesium is magnesium glycinate and this is combined with a secondary form such as magnesium acetyl taurate.

In alternate embodiments, the composition disclosed herein includes acetylcysteine, ascorbic acid, caffeine, methylcobalamin, pyridoxal-5-phosphate, riboflavin, magnesium glycinate, calcium L-5-methyl-tetrahydrofolate, milk thistle, panthenol, taurine, DHM dihydromyricetin, and/or niacinamide.

In other embodiments having acetylcysteine, also include bioeffective amounts of ascorbic acid, caffeine, methylcobalamin, pyridoxal-5-phosphate, riboflavin, magnesium glycinate, calcium L-5-methyl-tetrahydrofolate, milk thistle, panthenol, taurine, DHM dihydromyricetin, and/or niacinamide are combined in one unit dose.

Some embodiments having acetylcysteine also include bioeffective amounts of ascorbic acid, caffeine, methylcobalamin, pyridoxal-5-phosphate, riboflavin, magnesium glycinate, calcium L-5-methyl-tetrahydrofolate, milk thistle, panthenol, taurine, DHM dihydromyricetin, and/or niacinamide are provided in the form of a tablet, pill, capsule, lozenge, chewing gum, and/or transdermal patch.

In some embodiments, the dihydromyricetin is provided in an amount from about 50 mg to about 1,200 mg or from about 150 mg to about 600 mg. In some embodiments, the acetylcysteine is provided in an amount from about 70 mg to about 3,000 mg or from 250 mg to about 1,000 mg.

In various embodiments, the composition further includes caffeine. Preferably, the caffeine is provided in an amount from about 10 mg to about 200 mg or about 25 mg to about 100 mg.

In various embodiments, the composition further includes magnesium glycinate. Magnesium glycinate is the magnesium salt of glycine, an amino acid, and increases magnesium levels in the body. This chelated form of magnesium—where magnesium is bound to glycine—enhances bioavailability compared to other magnesium supplements, allowing for better absorption in the intestines with reduced risk of gastrointestinal side effects such as diarrhea or nausea, which are common with non-chelated forms like magnesium oxide.

In some embodiments, the composition further includes milk thistle. In some embodiments, the milk thistle is provided in an amount up to about 420 mg or from about 100 mg to about 400 mg.

In some embodiments, the composition further includes at least one vitamin and at least one amino acid. In some embodiments, the at least one vitamin comprises one or more B vitamins and vitamin C.

In some embodiments, the vitamin C is provided in an amount from about 50 mg to about 500 mg or from about 100 mg to about 400 mg. In some embodiments, the one or more B vitamins are selected from the group consisting of methylcobalamin, pyridoxal-5-phosphate, riboflavin, calcium L-5-methyl-tetrahydrofolate, panthenol, and niacinamide.

In some embodiments, the methylcobalamin is provided in an amount from about 1 mcg to about 5000 mcg or from about 250 mcg to about 1,000 mcg. In some embodiments, the pyridoxal-5-phosphate is provided in an amount from about 1 mg to about 600 mg or from about 2 mg to about 8 mg. In some embodiments, the riboflavin is provided in an amount from about 0.1 mg to about 400 mg or from about 1 mg to about 4 mg. In some embodiments, the calcium L-5-methyl-tetrahydrofolate is provided in an amount up to about 1 mg or from about 0.1 mg to about 0.5 mg. In some embodiments, the panthenol is provided in an amount up to about 1200 mg, or from about 2 mg to about 8 mg. In some embodiments, the niacinamide is provided in an amount up to about 50 mg or from about 7.5 mg to about 40 mg. In some embodiments, the one or more B vitamins consist of methylcobalamin, pyridoxal-5-phosphate, riboflavin, calcium L-5-methyl-tetrahydrofolate, panthenol, and niacinamide.

Some embodiments disclosed herein relate to methods of preventing, inhibiting, reducing, and/or shortening the duration of symptoms associated with consumption of alcohol and/or hangover. In other embodiments, the methods include administering an effective amount of the composition to a subject prior to, during, or after consumption of alcohol.

In some embodiments, the compositions disclosed herein are administered prior to consumption of alcohol. For example, in some embodiments, the composition is administered at about 8 hours prior to consumption of alcohol, or at about 6 hours prior to consumption of alcohol or at about 4 hours prior to consumption of alcohol, or at about 2 hours prior to consumption of alcohol, or about 1 hour prior to consumption of alcohol. In some embodiments, the composition is administered approximately 60 minutes prior to consumption of alcohol, or approximately 45 minutes prior to intake of alcohol, or about 30 minutes prior to intake of alcohol, or about 15 minutes prior to intake of alcohol, or about 5 minutes prior to intake of alcohol.

In some embodiments, the composition is administered during alcohol consumption. For example, in some embodiments, the composition is administered during consumption of an alcoholic beverage.

In other embodiments, the composition is administered after alcohol consumption. For example, in some embodiments, the composition is administered at about 8 hours after consumption of alcohol, or about 6 hours after consumption of alcohol, or about 4 hours after consumption of alcohol, or about 2 hours after consumption of alcohol, or about 1 hour after consumption of alcohol. In some embodiments, the composition is administered at about 60 minutes after intake of alcohol, or about 45 minutes after intake of alcohol, or about 30 minutes after intake of alcohol, or about 15 minutes after intake of alcohol, or about 5 minutes after intake of alcohol. In some embodiments, the composition is administered before bed after alcohol consumption.

In some embodiments, a single dose is 1˜4 units or more. In some embodiments, a single dose is 1-2 units, 1-3 units, 2-3 units, 2-4 units, or 3-4 units.

In some embodiments, the composition comprises acetylcysteine, also known as N-acetylcysteine (NAC). Acetylcysteine is an antioxidant and glutathione inducer indicated for mucolytic therapy and the treatment of acetaminophen overdose. In some embodiments, acetylcysteine is provided in a dosage range from about 70 mg to 3,000 mg, or from about 100 mg to about 2,000 mg, or from about 150 mg to about 1,750 mg, or from about 200 mg to about 1,500 mg per dose, or from about 250 mg to about 2,500 mg, or from about 500 mg to about 2,000 mg, or from about 750 mg to about 1,500 mg, or from about 500 mg to about 1,000 mg. In some embodiments, acetylcysteine is provided from about 250 mg to about 1,000 mg, or from about 500 mg to about 1,000 mg, or from about 750 mg to about 1,000 mg, or from about 250 mg to about 500 mg, or from about 250 mg to about 750 mg per dose. In some embodiments, acetylcysteine is provided at about 70 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, about 1,000 mg, about 1,250 mg, about 1,500 mg, about 1,750 mg, about 2,000 mg, about 2,250 mg, about 2,500 mg, about 2,750 mg, or about 3,000 mg per dose.

In some embodiments, the composition includes ascorbic acid. Ascorbic acid (Vitamin C) is a water-soluble vitamin found in citrus and other fruits and vegetables. Ascorbic acid is used to prevent and treat scurvy and is an essential nutrient involved in the repair of tissue, the formation of collagen, and the enzymatic production of certain neurotransmitters. In some embodiments, ascorbic acid is provided in a dosage range from about 50 mg to 500 mg, or from about 60 mg to about 475 mg, or from about 75 mg to about 450 mg, or from about 80 mg to about 420 mg, or from about 100 mg to 500 mg, or from about 200 mg to about 400 mg, or from about 250 mg to about 350 mg. In some embodiments, ascorbic acid is provided from about 100 mg to about 400 mg, or from about 200 mg to about 400 mg, or from about 300 mg to about 400 mg, or from about 100 mg to about 300 mg, or from about 100 mg to 200 mg per dose. In some embodiments, ascorbic acid is provided at about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg per dose.

In some embodiments, the composition includes caffeine. Caffeine is a bitter substance that occurs naturally in more than 60 plants including coffee beans, tea leaves, kola nuts, and cacao pods. Caffeine stimulates the central nervous system, is a diuretic, increases the release of acid in your stomach, may interfere with the absorption of calcium in the body, and increases blood pressure. In some embodiments, caffeine is provided in a dosage range from about 10 mg to 200 mg, or from about 15 mg to about 150 mg, or from about 20 mg to about 125 mg, or from about 25 mg to about 75 mg, or from about 50 mg to about 100 mg, or from about 75 mg to about 200 mg, or from about 50 mg to about 150 mg. In some embodiments, caffeine is provided from about 25 mg to about 100 mg, or from about 50 mg to about 75 mg, or from about 50 mg to 100 mg, or from about 25 mg to about 75 mg per dose. In some embodiments, caffeine is provided at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 125 mg, about 150 mg, about 175 mg, at about 200 mg per dose.

In some embodiments, the composition includes methylcobalamin, a naturally occurring form of Vitamin B12. Methylcobalamin is a water-soluble vitamin involved in red blood cell production, brain health, and DNA synthesis. In some embodiments, methylcobalamin is provided in dosage range from about 1 mcg to about 5000 mcg, or from about 25 mcg to 4,000 mcg, or from about 50 mcg to about 3,000 mcg, or from about 100 mcg to about 2,000 mcg, or from about 200 mcg to about 1,500 mcg, or from about 225 mcg to about 1,250 mcg, or from about 250 mcg to 750 mcg, or from about 500 mcg to about 1,000 mcg, or from about 750 mcg to about 2,000 mcg. In some embodiments, methylcobalamin is provided from about 250 mcg to about 1,000 mcg, or from about 500 mcg to about 1,000 mcg, or from about 500 mcg to about 750 mcg, or from about 250 mcg to about 750 per dose. In some embodiments, methylcobalamin is provided at about 1 mcg, about 25 mcg, about 50 mcg, about 75 mcg, about 100 mcg, about 125 mcg, about 150 mcg, about 175 mcg, about 200 mcg, about 225 mcg, about 250 mcg, about 275 mcg, about 300 mcg, about 325 mcg, about 350 mcg, about 375 mcg, about 400 mcg, about 425 mcg, about 450 mcg, about 475 mcg, about 500 mcg, about 600 mcg, about 700 mcg, about 800 mcg, about 900 mcg, about 1,000 mcg, about 1,250 mcg, about 1,500 mcg, about 1,750 mcg, about 2,000 mcg, about 2,500 mcg, or about 3,000 mcg per dose.

In some embodiments, the composition includes pyridoxal-5-phosphate (PSP), the active form of Vitamin B6. Pyridoxal-5-phosphate is a coenzyme in a variety of enzymatic reactions. In some embodiments, pyridoxal-5-phosphate is provided in a dosage range from about 1 mg to about 600 mg, or from about 1.25 mg to about 500 mg, or from about 1.5 to about 250 mg, or from about 1.75 mg to about 100 mg, or from about 1 mg to about 50 mg, or from about 1.25 to about 25 mg, or from about 1.5 to about 10 mg, or from about 2 mg to about 6 mg, or from about 4 mg to about 8 mg, or from about 100 mg to about 550 mg, or from about 150 mg to about 450 mg, or from about 200 mg to about 300 mg. In some embodiments, pyridoxal-5-phosphate is provided from about 2 mg to about 8 mg, or from about 2 mg to about 6 mg, or from about 4 mg to about 8 mg, or from about 6 mg to about 8 mg per dose. In some embodiments, pyridoxal-5-phosphate is provided at about 1 mg, about 2 mg, about 3 mg, about 4 mg, about 5 mg, about 6 mg, about 7 mg, about 8 mg, about 9 mg, about 10 mg, about 20 mg, about 25 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg per dose.

In some embodiments, the composition includes riboflavin (Vitamin B2). Riboflavin is one of the B vitamins.

B vitamins help the body to convert food (carbohydrates) into fuel (glucose), which is used to produce energy. In some embodiments, riboflavin is provided in a dosage range from about 0.1 mg to about 400 mg, or from about 0.5 mg to about 250 mg, or from about 0.75 to about 150 mg, or from about 1 to about 100 mg, or from about 0.75 mg to about 50 mg, or from about 0.5 to about 10 mg, or from about 2 mg to about 4 mg, or from about 1 mg to about 3 mg, or from about 10 mg to about 100 mg, or from about 25 mg to about 75 mg, or from about 15 mg to about 50 mg. In some embodiments, riboflavin is provided in a range from about 1 mg to about 4 mg, or from about 2 mg to about 4 mg, or from about 3 mg to about 4 mg, or from about 1 mg to about 3 mg per dose. In some embodiments, riboflavin is provided at about 0.5 mg, about 1 mg, about 1.5 mg, about 2 mg, about 2.5 mg, about 3 mg, about 3.5 mg, about 4 mg, about 4.5 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg per dose.

In some embodiments, the composition includes Calcium L-5-Methyltetrahydrofolate, a member of the folate group of vitamins (Vitamin B9). Folate is a water-soluble Vitamin B naturally found in food sources. It helps in forming red blood cells and treats low folate levels and anemia. In some embodiments, calcium L-5-methyl-tetrahydrofolate is provided at a dosage amount of up to about 1 mg, or up to about 0.75 mg, or up to about 0.5 mg, or up to about 0.25 mg, or up to about 0.15 mg, or up to about 0.1 mg, or up to about 0.05 mg. In some embodiments, calcium L-5-methyl-tetrahydrofolate is provided in a range from about 0.1 mg to about 0.5 mg, or from about 0.1 mg to about 0.25 mg, or from about 0.2 mg to about 0.5 mg, or from about 0.1 mg to about 0.4 mg per dose. In some embodiments, calcium L-5-methyl-tetrahydrofolate is provided at about 0.1 mg, about 0.2 mg, about 0.3 mg, about 0.4 mg, about 0.5 mg, about 0.6 mg, about 0.7 mg, about 0.8 mg, about 0.9 mg, or about 1 mg per dose.

In some embodiments, the composition includes milk thistle (silymarin). Milk thistle is a flowering herb related to the daisy and ragweed family and is promoted as a dietary supplement for hepatitis, cirrhosis, jaundice, diabetes, indigestion, and other conditions. In some embodiments, milk thistle is provided at a dosage amount of up to about 420 mg milk thistle, or up to about 400 mg, or up to about 350 mg, or up to about 300 mg, or up to about 250 mg, or up to about 200 mg, or up to about 150 mg, or up to about 100 mg, or up to about 50 mg. In some embodiments, milk thistle is provided in a range from about 100 mg to about 400 mg, or from about 100 mg to about 300 mg, or from about 200 mg to about 400 mg, or from about 200 mg to about 300 mg per dose. In some embodiments, milk thistle is provided at about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, or about 420 mg per dose.

In some embodiments, the composition includes panthenol. Panthenol (also called pantothenol) is the alcohol analog of pantothenic acid (Vitamin B5) and is thus a provitamin of B5. In organisms, it is quickly oxidized to pantothenic acid. In some embodiments, panthenol is provided in a dosage amount of up to about 1,200 mg panthenol, or up to about 1,000 mg, or up to about 750 mg, or up to about 500 mg, or up to about 250 mg, or up to about 100 mg, or up to about 50 mg, or up to about 25 mg, or up to about 15 mg, or up to about 10 mg, or up to about 5 mg. In some embodiments, panthenol is provided from about 2 mg to about 8 mg, or from about 2 mg to about 6 mg, or from about 4 mg to about 8 mg, or from about 6 mg to about 8 mg per dose. In some embodiments, panthenol is provided at about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 50 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1,000 mg, about 1,100 mg, or about 1,200 mg, per dose.

In some embodiments, the composition is completely free of taurine. In other embodiments, the composition is free from isolated and free taurine, which can cause nausea, but may include neuroprotective bound forms.

In some embodiments, the composition includes Dihydromyricetin (DHM), also known as Ampelopsin. DHM is a flavanonol, a type of flavonoid, extract from the Japanese Raisin Tree.

In some embodiments, the composition comprises Dihydromyricetin (DHM) in a range from about 50 mg to about 1,200 mg, or from about 75 mg to about 1,000 mg, or from about 100 mg to 750 mg, or from about 125 mg to about 700 mg. In some embodiments, dihydromyricetin is provided in a range from about 150 mg to about 600 mg, or from about 300 mg to about 600 mg, or from about 450 mg to about 600 mg, or from about 150 mg to about 450 mg, or from 150 mg to about 300 mg, or from about 450 mg to about 600 mg per dose. In some embodiments, dihydromyricetin is provided at about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg per dose.

In some embodiments, the composition includes niacinamide or niacin, also called nicotinamide. Niacinamide is a form of vitamin B3 found in many foods including meat, fish, milk, eggs, green vegetables, and cereals. Niacinamide is required for the function of fats and sugars in the body and to maintain healthy cells. In some embodiments, the composition comprises up to about 50 mg niacinamide, or up to about 40 mg, or up to about 30 mg, or up to about 20 mg, or up to about 10 mg, or up to about 5 mg.

In some embodiments, niacinamide is provided in a range from about 7.5 mg to about 40 mg, or from about 15 mg to about 40 mg, or from about 25 mg to about 40 mg, or from about 7.5 mg to about 30 mg, or from about 7.5 mg to about 20 mg, or from about 15 mg to about 30 mg per dose. In some embodiments, niacinamide is provided at about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg per dose.

Dosage ranges are summarized in Table 1 below.

TABLE 1

| | Minimum | Maximum |
|---|---|---|
| Substance Name | Dose | Dose |
| Acetylcysteine | 70 mg | 3,000 mg |
| Ascorbic Acid | 50 mg | 500 mg |
| Caffeine | 10 mg | 200 mg |
| Methylcobalamin | 1 mcg | 5,000 mcg |
| Pyridoxal-5-phosphate | 1 mg | 600 mg |
| Riboflavin | 0.1 mg | 400 mg |
| Magnesium glycinate | | 300 mg |
| Calcium L-5-methyl-tetrahydrofolate | | 1 mg |
| Milk thistle | | 420 mg |
| Panthenol | | 1,200 mg |
| Magnesium acetyl taurate | 20 mg | 1,000 mg |
| Dihydromyricetin | 50 mg | 1,200 mg |
| Niacinamide | | 50 mg |

In some embodiments, the composition further comprises one or more cannabinoids in a range from about 2 mg to about 2,000 mg, or from about 5 mg to about 1,500 mg, or from about 10 mg to about 1,000 mg, or from about 25 mg to about 500 mg, or from about 50 mg to 100 mg. In some embodiments, the one or more cannabinoids are provided at about 2 mg, about 5 mg, about 10 mg, about 15 mg, about 20 mg, about 25 mg, about 50 mg, about 100 mg, about 125 mg, about 150 mg, about 175 mg, about 200 mg, about 250 mg, about 300 mg, about 350 mg, about 400 mg, about 450 mg, about 500 mg, about 600 mg, about 700 mg, about 800 mg, about 900 mg, about 1,000 mg, about 1,250 mg, about 1,500 mg, about 1,750 mg, or about 2,000 mg.

Hundreds of cannabinoids are currently known in the art, including, for example, but not limited to, cannabidiol (CBD), tetrahydrocannabivarin (THCV), cannabidivarin (CBDV), cannabigerol (CBG), cannabichromene (CBC), cannabinol (CBN), tetrahydrocannabinolic acid (THCA), cannabidiolic acid (CBDA), and cannabigerolic acid (CBGa), and the like.

Hangover symptoms typically develop when BAC begins to decline, typically when BAC returns to substantially about zero. In the methods disclosed herein, the compositions disclosed herein are administered or taken prior to the onset of symptoms. For example, administration can be about 120 minutes before alcohol intake, or about 90 minutes before alcohol intake, or about 60 minutes before alcohol intake, or about 30 minutes before alcohol intake, or about 20 minutes before alcohol intake, or immediately prior to alcohol intake, or simultaneously with alcohol intake. In some embodiments, the compositions disclosed herein can be administered after alcohol consumption and prior to onset of symptoms, for example, the composition can be administered or taken up to about 1 hour, or up to about 2 hours, or up to about 3 hours, or up to about 4 hours after alcohol intake, or up to about 6 hours, or up to about 8 hours after alcohol consumption. In some embodiments, the compositions disclosed herein can be administered at the onset of hangover symptoms.

The compositions disclosed herein can be administered by methods known in the art. For example, the pharmaceutical composition can be administered systemically. For the purposes of this specification, “systemic administration” means administration to a human by a method that causes the compositions to be absorbed into the bloodstream by any method known in the art.

The compositions can be administered in the form of tablets, including, e.g., orally dissolvable tablets, chewable tablets, capsules, lozenges, pills, troches, elixirs, suspensions, syrups, wafers, chewing gum, strips, films (e.g., orally-dissolving thin films), soluble powders, effervescent compositions, and the like.

In some embodiments, the compositions disclosed herein can be administered enterally or parenterally, e.g., intravenously, intramuscularly, subcutaneously, as injectable solutions or suspensions, intraperitoneally, sublingually, or rectally (e.g., by suppositories). Administration can also be intranasally, in the form of, for example, an intranasal spray, or transdermally, in the form of a transdermal patch.

In some embodiments, the compositions disclosed herein can be formulated as pharmaceutical preparations, optionally, with a suitable pharmaceutical carrier (vehicle) or excipient, as understood by practitioners in the art. These preparations can be made according to conventional chemical methods.

In some embodiments, for example, in the case of tablets for oral use, carriers commonly used include lactose and corn starch, and lubricating agents such as magnesium stearate are commonly added. For oral administration in capsule form, useful carriers include lactose and corn starch. Further examples of carriers and excipients include milk, sugar, certain types of clay, gelatin, stearic acid or salts thereof, calcium stearate, talc, vegetable fats or oils, gums and glycols.

When aqueous suspensions are used for oral administration, emulsifying and/or suspending agents are commonly added. In addition, sweetening and/or flavoring agents may be added to the oral compositions. For intramuscular, intraperitoneal, subcutaneous and intravenous use, sterile solutions of the pharmaceutical compositions can be employed, and the pH of the solutions can be suitably adjusted and buffered. For intravenous use, the total concentration of the solute(s) can be controlled in order to render the preparation isotonic.

In some embodiments, the composition is provided in pill or tablet or capsule form and includes an acetylcysteine, ascorbic acid, caffeine, methylcobalamin, pyridoxal-5-phosphate, riboflavin, magnesium glycinate, calcium L-5-methyl-tetrahydrofolate, milk thistle, panthenol, taurine, DHM dihydromyricetin, and/or niacinamide, with or without a taste masking ingredients, diluents, etc. The pill can be formulated by a number of techniques known to a skilled artisan. The pill or tablet or capsule can be administered with water.

The compositions of the invention disclosed herein can further comprise one or more pharmaceutically acceptable additional ingredient(s) such as alum, stabilizers, buffers, coloring agents, flavoring agents, and the like. In some embodiments, orally administered pharmaceutical compositions can further include breath neutralizers, e.g., peppermint or menthol scents.

In some embodiments, the compositions disclosed herein can be administered by controlled release. Controlled release administration is a method of drug delivery to achieve a certain level of the drug over a particular period of time. The level typically is measured by plasma concentration. Methods for controlled release of drugs are well known in the art. In some embodiments, the compositions disclosed herein can be formulated for controlled release. For example, in some embodiments, the compositions can be a capsule containing beadlets, wherein some of the beadlets dissolve instantaneously and some of the beadlets dissolve at delayed times due to different types of beadlet coatings.

In some embodiments, the compositions disclosed herein comprise an active ingredient, wherein the active ingredient includes: acetylcysteine, ascorbic acid, caffeine, methylcobalamin, pyridoxal-5-phosphate, riboflavin, magnesium glycinate, calcium L-5-methyl-tetrahydrofolate, milk thistle, panthenol, taurine, DHM dihydromyricetin, and niacinamide.

In some embodiments, the compositions disclosed herein include: a) acetylcysteine, b) ascorbic acid, c) caffeine, d) methylcobalamin, e) pyridoxal-5-phosphate, f) riboflavin, g) magnesium glycinate, h) calcium L-5-methyl-tetrahydrofolate, i) milk thistle, j) panthenol, k) DHM dihydromyricetin, and l) niacinamide, and m) at least one carrier and/or excipient.

By excluding free form taurine, the composition avoids a common source of nausea in prior remedies. Instead, bound forms like magnesium acetyl taurate provide taurine-related benefits (e.g., soothing muscles, neuroprotection, hydration) without nausea. This form crosses the blood-brain barrier, offering protection against veisalgia's neurological effects.

### Example 1

The following examples demonstrate that hangover symptoms are reduced or prevented when using the compositions and methods disclosed herein. 300 units of the composition disclosed herein were formulated as shown in the table 2 below.

TABLE 2

| Ingredient | Calc Quantity | Units |
|---|---|---|
| Acetylcysteine USP | 75 | gm |
| Ascorbic Acid USP FinePowder | 30 | gm |
| Caffeine USP Anhydrous | 7.5 | gm |
| Methylcobalamin | 0.075 | gm |
| Riboflavin USP (Vitamin B2) | 0.3 | gm |
| Pyridoxal-5-PhosphateMonohydrate | 0.6 | gm |
| Magnesium Glycinate (15% Magnesium) | 8.1 | gm |
| Calcium L-5-Methyl-Tetrahydrofolate USP | 0.0345 | gm |
| Milk Thistle (SilybumMarianum) | 30 | gm |
| Panthenol USP (Dl) | 0.6 | gm |
| Taurine | 37.5 | gm |
| DHM Dihydromyricetin | 45 | gm |
| Niacinamide USP | 2.7 | gm |

### Example 2

Capsule for oral delivery, wherein the dosage for an average adult is 1-2 capsules is another example. The capsule contains a proprietary blend of acetylcysteine, Dihydromyricetin, Milk Thistle Extract and caffeine. The proprietary blend optionally contains taurine in one embodiment and no taurine in another embodiment. In Example 2, various vitamins and minerals are included so that one capsule further contains: 100 mg of Vitamin C (Ascorbic Acid), 1 mg Riboflavin, 4 mg Niacin, 2 mg Vitamin B6, 100 mcg Folate, 250 mcg Vitamin B12, 2 mg Pantothenic Acid, and 4 mg Magnesium (Glycinate).

While there have been described what are presently believed to be the preferred embodiments of the present invention, other and further embodiments, modifications, and improvements will be known to those skilled in the art, and it is intended to include all such further embodiments, modifications, and improvements come within the true scope of the invention described herein.

What is claimed is:

1. An orally deliverable composition for reducing veisalgia symptoms and nausea in a subject, comprising:
dihydromyricetin, acetylcysteine, and an antiemetic that decreases smooth muscle contraction in the digestive tract;
the antiemetic is selected from the group consisting of phosphoric acid, ginger, peppermint, lemon, bismuth subsalicylate, and combinations thereof in an amount sufficient to decrease smooth muscle contraction and inhibit nausea in the subject;
the composition is packaged as a viscous liquid, capsule, tablet or chewable form for oral administration; and
the composition contains no free form taurine amino acid to avoid taurine-induced nausea.

2. The composition of claim 1, further comprising at least one neuroprotective form of taurine such as magnesium acetyl taurate which is not known to cause nausea in subjects and which can pass the blood brain barrier to protect the brain and neurons from some effects of veisalgia.

3. The composition of claim 1, wherein and
the composition includes at least one neuroprotective form of taurine such as magnesium acetyl taurate which is not known to cause nausea, and which can pass the blood brain barrier to enable neuroprotection from veisalgia; and
magnesium glycinate.

4. The composition of claim 1, wherein the dihydromyricetin is provided in an amount from between 150 mg to about 600 mg; and
the acetylcysteine is provided in an amount from about 70 mg to about 3,000 mg.

5. The composition of claim 4, wherein the acetylcysteine is provided in an amount from about or from 250 mg to about 1,000 mg.

6. The composition of claim 1, further comprising magnesium, the magnesium is selected from the group consisting of: magnesium oxide, magnesium citrate, magnesium glycinate, magnesium acetyl taurate, magnesium sulfate, magnesium L-Threonate, magnesium salts, and combinations thereof.

7. The composition of claim 6, wherein the magnesium includes magnesium acetyl taurate, which is not liposomally encapsulated to sooth cramped muscles.

8. The composition of claim 6, wherein the magnesium includes magnesium acetyl taurate, which is liposomally encapsulated to offer neuroprotection to the brain and to bypass the upper digestive tract to minimize any nausea.

9. The composition of claim 6, wherein the magnesium includes magnesium glycinate in an amount between 25 mg and 300 mg, wherein the magnesium glycinate is a chelated form that enhances bioavailability and reduces gastrointestinal side effects, and wherein the magnesium acts as a cofactor in over 300 enzymatic reactions, facilitating ATP synthesis, regulating ion channels for muscle and nerve function, and replenishing alcohol-depleted magnesium stores to alleviate symptoms such as muscle cramps, fatigue, headaches, and irritability.

10. The composition of claim 7, wherein the magnesium includes magnesium acetyl taurate to increase brain tissue levels of magnesium and to provide neuroprotection to brain tissue without nausea, which can be induced by free form taurine supplementation.

11. The composition of claim 8, wherein the milk thistle is provided in an amount up to about 420 mg.

12. The composition of claim 9, further comprising:
milk thistle, which is provided in an amount between 100 mg to 400 mg.

13. The composition of claim 10, further comprising:
one or more B vitamins and vitamin C.

14. The composition of claim 13, wherein the one or more B vitamins are selected from the group consisting of methylcobalamin, pyridoxal-5-phosphate, riboflavin, calcium L-5-methyl-tetrahydrofolate, panthenol, niacinamide, and combinations thereof.

15. The composition of claim 1, wherein the composition is completely free of taurine, and wherein the magnesium cooperates with the dihydromyricetin and acetylcysteine to replenish alcohol-depleted magnesium stores, promote cellular hydration, reduce inflammation, and alleviate veisalgia symptoms without any taurine-related nausea.

16. The composition of claim 15, wherein the magnesium includes magnesium glycinate as a primary source in an amount between 25 mg and 300 mg, the magnesium glycinate being a chelated form that enhances bioavailability and reduces gastrointestinal side effects, and wherein the magnesium acts as a cofactor in over 300 enzymatic reactions, facilitating ATP synthesis, regulating ion channels for muscle and nerve function, and supporting recovery from alcohol-induced depletion.

17. The composition of claim 1, wherein the composition is free from isolated and free form taurine to avoid taurine-induced nausea but includes neuroprotective bound forms of taurine, and wherein the magnesium cooperates with the bound taurine forms to provide neuroprotection, soothe muscles, improve cellular hydration, and alleviate veisalgia symptoms without inducing nausea.

18. The composition of claim 17, wherein the bound form of taurine is magnesium acetyl taurate in an amount from 20 mg to 1,000 mg and the magnesium acetyl taurate is liposomally encapsulated to enhance brain delivery and bypass the upper digestive tract.

19. The composition of claim 17, wherein the magnesium includes magnesium glycinate as a primary source in an amount between 25 mg and 300 mg, the magnesium glycinate being a chelated form that enhances bioavailability and reduces gastrointestinal side effects, and wherein the magnesium acts as a cofactor in over 300 enzymatic reactions, facilitating ATP synthesis, regulating ion channels for muscle and nerve function, and supporting recovery from alcohol-induced depletion in cooperation with the bound taurine forms.

20. The composition of claim 17, further comprising milk thistle in an amount between 100 mg and 400 mg, one or more B vitamins, and vitamin C, wherein the magnesium cooperates with these components to reduce hangover symptoms by at least 10% to 100%.
